(19) United States
(12) Patent Application Publication
(10) Pub. No.: US 2023/0207098 A1
(43) Pub. Date: Jun. 29, 2023

(54) VIBRATORY OUTPUT HEALTH DEVICE

(71) Applicant: Luvo LLC, Ripon, WI (US)

(72) Inventor: Sanjib Nandi, Berlin, WI (US)

(73) Assignee: Luvo LLC, Ripon, WI (US)

(21) Appl. No.: 17/645,764

(22) Filed: Dec. 23, 2021

(51) Int. Cl. G16H 20/40; G16H 20/60; G16H 20/70; G16H 20/30; G16H 10/20

(52) U.S. Cl. CPC G16H 20/40; G16H 20/60; G16H 20/70; G16H 20/30; G16H 10/20

(57) ABSTRACT

A method for improving health of a user includes receiving an answer to a question from the user, identifying a first category of therapy associated with the answer to the question, and displaying a visual indicator associated with the first category to the user. Outputting guidance for the user including tasks operative to improve the health of the user, the guidance including a vibratory output associated with the first category operative to improve the health of the user.

### BACKGROUND

The present invention relates to wellness. Wellness is a condition of good physical and mental health. Physical and mental health may be improved in a person by many factors including, exercise, meditation, water intake and monitoring sleep patterns.

Regarding meditation, studies have shown that regular meditation has a variety of positive wellness effects.

### SUMMARY

Embodiments are directed to a method for improving the health of a user, which includes receiving an answer to a question from the user, identifying a first category of therapy associated with the answer to the question, and displaying a visual indicator associated with the first category to the user. Outputting guidance for the user including tasks operative to improve the health of the user, the guidance including a vibratory output associated with the first category operative to improve the health of the user.

A system for improving health of a user, the system comprising includes a memory, a display, and a processor operative to receive an answer to a question from the user and identify a first category of therapy associated with the answer to the question. The processor is further operative to display a visual indicator associated with the first category to the user, and output guidance for the user including tasks operative to improve the health of the user, the guidance including a vibratory output associated with the first category operative to improve the health of the user.

A method for improving the health of a user comprises receiving an answer to a question from the user, identifying a first category of therapy associated with the answer to the question, displaying a visual indicator associated with the first category to the user, the visual indicator including a color recommendation of a diet for the user, and a meditative therapy recommendation that includes vibrational therapy at about 100 Hz to 250 Hz, and outputting guidance for the user including tasks operative to improve the health of the user, the guidance including a vibratory and auditory output associated with the first category operative to improve the health of the user.

### BRIEF DESCRIPTION OF THE DRAWINGS

FIG. 1 illustrates a block diagram of a system.

FIG. 2 illustrates a block diagram of a method of operation of the system of FIG. 1.

FIG. 3 shows a block diagram of a method that may be performed by the system of FIG. 1.

FIG. 4 illustrates a graphical user interface.

FIG. 5 illustrates a graphical user interface.

FIG. 6 illustrates a graphical user interface.

FIG. 7 illustrates a graphical user interface.

FIG. 8 illustrates a graphical user interface.

FIG. 9 illustrates a graphical user interface.

FIG. 10 illustrates a graphical user interface.

FIG. 11 illustrates a graphical user interface.

FIG. 12 illustrates a graphical user interface.

FIG. 13 illustrates a graphical user interface.

FIG. 14 illustrates a graphical user interface.

FIG. 15 illustrates a graphical user interface.

FIG. 16 illustrates a graphical user interface.

FIG. 17 illustrates a graphical user interface.

FIG. 18 illustrates a graphical user interface.

FIG. 19 illustrates a graphical user interface.

### DETAILED DESCRIPTION

The wellness in a person is a condition of good mental and physical health. To be well in this regard, a person should have a healthy diet, water intake and exercise and perform meditation to improve one's wellness.

However, a challenge for people interested in improving wellness is that they do not drink enough water. Our brain and body are composed of 60 to 70 percent water. It is one of the basic elements required for human survival. Further, meditation and exercise should also be tailored to improve wellness, thus improving their wellness. The system described herein may determine whether a user should increase their water intake. The system may remind a user to drink water over a periods of time to increase their water intake and reach for example, a daily or weekly water intake goal.

Parts of being well or healthy may include several factors such as, for example, heart rate, resting heart rate, blood pressure, mood, alertness, contentment, gratefulness, physical health, body fat percentage, sleep and brain activity.

If a person would like to improve any of the above-mentioned factors, a regimen of meditation, exercise and diet and water intake directed to improving the health of a user may be beneficial. Examples of such a regimen include identifying the type of tasks such as meditation and developing a prescription for improving the health of the user. A prescription associated with the health of a user may include, for example, a particular exercise regimen, water intake per day, exercise, and meditation among other things.

It is desirable for a device to be operative to assist a user in determining a regimen of exercise, water intake, and meditation tailored to address the needs of the user and improve the health of the user.

Meditation is often a strong component of a prescription to improve health thus, effective meditation may help to improve the health of a user. For example, meditating with sounds or music may improve the effectiveness of the mediation.

Research has shown that after about eight weeks of meditation, the electrical activity in the amygdala (an almond-shaped structure in the brain responsible for processing our emotions) demonstrated far less activity, correlating with reduced anxiety, worry and fear.

Meditation has been shown to balance the corpus callosum, the nerve bundle cross-linking the two hemispheres of the brain, making it stronger, thicker and more neurologically connected. This adds to total brain thickness. The benefits of the whole brain thickness are better focus, excellent mental health and deeper and clearer thinking.

Vibration therapy can improve the well-being of the person. The therapy may include targeted vibration therapy focus on particular part of the body such as the palm of the user or whole-body vibration.

The body experiences several whole-body vibrations or targeted vibration that are operative to increase one's vibrational frequency. Studies have shown that providing a vibratory stimulation at a particular frequency and amplitude may increase dopamine levels in the brain, which often increases one's happiness.

In a study, participants underwent six passive whole body vibration sessions. The study found that two minutes passive whole body vibration sessions had positive effects on attention and inhibition in young adults.

In an experiment to differentiate the multiple vibration frequencies felt on the hand and found out that human sensitivity is optimal in lower frequency of around 250 Hz.

If the brain sensitivity is optimum at about 250 Hz as studies have shown than a vibratory stimulus through the palm of the user may improve the effectiveness of some meditation that is prescribed to improve the overall health of a user.

Such vibratory therapy appears to improve cognitive function by producing similar psychological responses as exercise including, for example, increased oxygen uptake and increased blood flow and may also boost energy levels. Thus, some of the beneficial aspects of exercise may be achieved by stimulating a person with vibrations of a particular or range of frequencies and amplitudes. Other effective techniques may include receiving auditory stimulation from music or sounds associated with improving one's mental and physical health.

In embodiments the vibratory therapy when accompanying with meditation can synergistically improve the benefits of meditation and improve the overall health of the user.

Other meditation practices such as, for example audible sounds and music may be prescribed in conjugation with vibration therapy for persons who desire to improve their manifestation for health, happiness or high performance.

Determining the type of mediation therapy, water intake and exercise tasks may be challenging for some users. Further, determining a prescription including, for example, exercise, water intake, sleep patterns, and meditation that is associated with and intended to improve health is also a challenge.

Embodiments described herein include a device, system, and method operative to determine and identify tasks that may be beneficial to a user and prescribe a regimen that improves the effectiveness of such tasks.

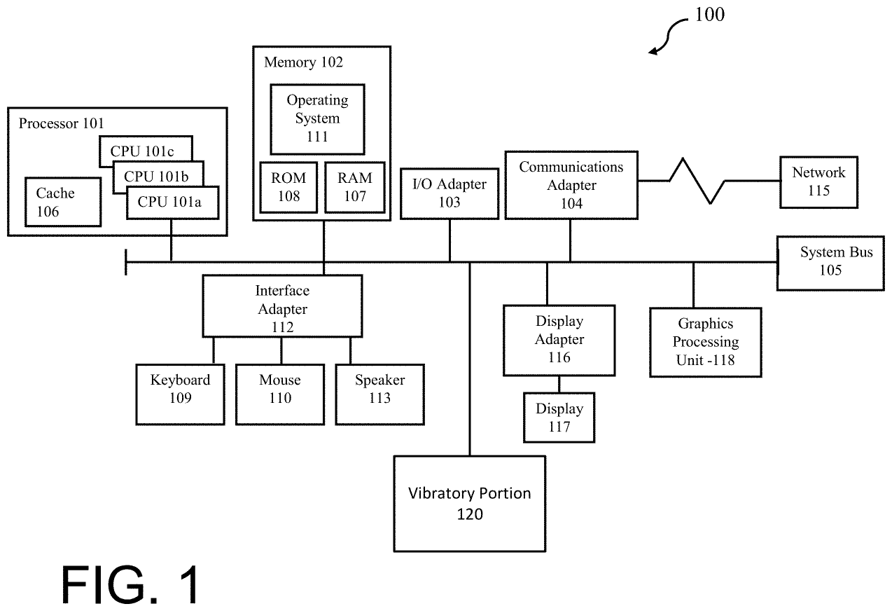

FIG. 1 illustrates a block diagram of an exemplary computing environment and computer system 100 for use in practicing the embodiments described herein. The environment and system described herein can be implemented in hardware, software (e.g., firmware), or a combination thereof. In an exemplary embodiment, a hardware implementation can include a microprocessor of a special or general-purpose digital computer, such as a personal computer, workstation, minicomputer, or mainframe computer. Computer 100 therefore can embody a general-purpose computer. In another exemplary embodiment, the implementation can be part of a mobile device, such as, for example, a mobile phone, a personal data assistant (PDA), a tablet computer, etc.

As shown in FIG. 1, the computer 100 includes processor 101. Computer 100 also includes memory 102 communicatively coupled to processor 101, and one or more input/output adapters 103 that can be communicatively coupled via system bus 105. Memory 102 can be communicatively coupled to one or more internal or external memory devices via a storage interface 108. Communications adapter 116 can communicatively connect computer 100 to one or more networks 106. System bus 105 can communicatively connect one or more user interfaces via input/output (I/O) adapter 103. I/O adapter 103 can connect a plurality of input devices 104 to computer 100. Input devices can include, for example, a keyboard, a mouse, a microphone, a sensor, etc. System bus 105 can also communicatively connect one or more output devices 107 via I/O adapter 103. Output device 107 can include, for example, a display, a speaker, a touchscreen, etc.

Processor 101 is a hardware device for executing program instructions (aka software), stored in a computer-readable memory (e.g., memory 102). Processor 101 can be any custom made or commercially available processor, a central processing unit (CPU), a plurality of CPUs, an auxiliary processor among several other processors associated with the computer 100, a semiconductor-based microprocessor (in the form of a microchip or chip set), or generally any device for executing instructions. Processor 101 can include a cache memory 122, which can include, but is not limited to, an instruction cache to speed up executable instruction fetch, a data cache to speed up data fetch and store, and a translation lookaside buffer (TLB) used to speed up virtual-to-physical address translation for both executable instructions and data. Cache memory 122 can be organized as a hierarchy of more cache levels (L1, L2, etc.).

Processor 101 can be disposed in communication with one or more memory devices (e.g., RAM 109, ROM 110, one or more external databases 121, etc.) via a storage interface 108. Storage interface 108 can also connect to one or more memory devices including, without limitation, one or more databases 121, and/or one or more other memory drives (not shown) including, for example, a removable disc drive, etc., employing connection protocols such as serial advanced technology attachment (SATA), integrated drive electronics (IDE), IEEE-1394, universal serial bus (USB), fiber channel, small computer systems interface (SCSI), etc. The memory drives can be, for example, a drum, a magnetic disc drive, a magneto-optical drive, an optical drive, a redundant array of independent discs (RAID), a solid-state memory device, a solid-state drive, etc. Variations of memory devices can be used for implementing, for example, list all databases from other figures.

Memory 102 can include random access memory (RAM) 109 and read only memory (ROM) 110. RAM 109 can be any one or combination of volatile memory elements (e.g., DRAM, SRAM, SDRAM, etc.). ROM 110 can include any one or more nonvolatile memory elements (e.g., erasable programmable read only memory (EPROM), flash memory, electronically erasable programmable read only memory (EEPROM), programmable read only memory (PROM), tape, compact disc read only memory (CD-ROM), disk, cartridge, cassette or the like, etc.). Moreover, memory 102 can incorporate electronic, magnetic, optical, and/or other types of non-transitory computer-readable storage media. Memory 102 can also be a distributed architecture, where various components are situated remote from one another, but can be accessed by processor 101.

The instructions in memory 102 can include one or more separate programs, each of which can include an ordered listing of computer-executable instructions for implementing logical functions. In the example of FIG. 1, the instructions in memory 102 can include an operating system 111. Operating system 111 can control the execution of other computer programs and provides scheduling, input-output control, file and data management, memory management, and communication control and related services.

The program instructions stored in memory 102 can further include application data 112, and for a user interface 113.

Memory can also include program instructions for Engine 1, configured to improve the wellness of a user.

I/O adapter 103 can be, for example but not limited to, one or more buses or other wired or wireless connections. I/O adapter 103 can have additional elements (which are omitted for simplicity) such as controllers, microprocessors, buffers (caches), drivers, repeaters, and receivers, which can work in concert to enable communications. Further, I/O adapter 103 can facilitate address, control, and/or data connections to enable appropriate communications among the aforementioned components.

I/O adapter 103 can further include a display adapter coupled to one or more displays. I/O adapter 103 can be configured to operatively connect one or more input/output (I/O) devices 107 to computer 100. For example, I/O 103 can connect a keyboard and mouse, a touchscreen, a speaker, a haptic output device, or other output device. Output devices 107 can include but are not limited to a printer, a scanner, and/or the like. Other output devices can also be included, although not shown. Finally, the I/O devices connectable to I/O adapter 103 can further include devices that communicate both inputs and outputs, for instance but not limited to, a network interface card (NIC) or modulator/demodulator (for accessing other files, devices, systems, or a network), a radio frequency (RF) or other transceiver, a telephonic interface, a bridge, a router, and the like.

According to some embodiments, computer 100 can include a mobile communications adapter 123. Mobile communications adapter 123 can include GPS, cellular, mobile, and/or other communications protocols for wireless communication.

In some embodiments, computer 100 can further include communications adapter 116 for coupling to a network 106.

Network 106 can be an IP-based network for communication between computer 100 and any external device. Network 106 transmits and receives data between computer 100 and devices and/or systems external to computer 100. In an exemplary embodiment, network 106 can be a managed IP network administered by a service provider. Network 106 can be a network internal to an aircraft, such as, for example, an avionics network, etc. Network 106 can be implemented in a wireless fashion, e.g., using wireless protocols and technologies, such as Wi-Fi, WiMAX, etc. Network 106 can also be a wired network, e.g., an Ethernet network, an ARINC 429 network, a controller area network (CAN), etc., having any wired connectivity including, e.g., an RS232 connection, R5422 connection, etc. Network 106 can also be a packet-switched network such as a local area network, wide area network, metropolitan area network, Internet network, or other similar type of network environment. The network 106 can be a fixed wireless network, a wireless local area network (LAN), a wireless wide area network (WAN) a personal area network (PAN), a virtual private network (VPN), intranet or other suitable network system.

Network 106 can operatively connect computer 100 to one or more devices including device 117, device 118, and device 120. Network 106 can also connect computer 100 to one or more servers such as, for example, server 119.

If computer 100 is a PC, workstation, laptop, tablet computer and/or the like, the instructions in the memory 102 can further include a basic input output system (BIOS) (omitted for simplicity). The BIOS is a set of routines that initialize and test hardware at startup, start operating system 111, and support the transfer of data among the operatively connected hardware devices. The BIOS is typically stored in ROM 110 so that the BIOS can be executed when computer 100 is activated. When computer 100 is in operation, processor 101 can be configured to execute instructions stored within the memory 102, to communicate data to and from the memory 102, and to generally control operations of the computer 100 pursuant to the instructions.

In embodiments the system 100 may include, for example, a smart phone or computer. The audio portion 114 may be a stand-alone device that is communicatively connected to the processor via a wired or wireless connection. In embodiments, the vibratory portion 120 may be integrated into a smart phone or may include a stand-alone vibratory device communicatively connected to the processor 102.

In operation, the system 100 is operative to present one or a series of questions related to the condition of the chakras of the user. Such questions may include, for example, whether the user feels safe and supported, whether the user feels inspired and passionate, the age and sex of the user, the mood of a user, the meditative experience of the user, and habitual sexual activity of a user, among others.

In embodiments. Different issues identified by the system may result in a prescription of tasks such as meditation which may be classified in frequencies.

After the user inputs answers to the questions, the system 100 is operative to determine whether the user may find meditation beneficial and what type of meditation therapy is recommended for the user.

In embodiments, the system 100 is operative to provide exercises such as, for example, breathing or physical exercises such as, for example, yoga. The system 100 may track several factors such as, for example, steps and distance traveled over a period of time, water intake for a period of time, heartrate, amount of sleep, and the mood of the user. In embodiments, each of these factors may be associated with a score that may be cumulated to determine an overall health score.

In embodiments the system 100 may request feedback from a user regarding the feelings of the user after following the prescription of exercise, meditation, gratitude and water intake for the day. In this regard, human vibration may be modulated by several other factors, including water intake and a time of the water intake that may be correlated to a mood of the user. Sleep patterns may also be a factor in the modulation of human vibration. The sleep may be correlated with time, sleep patterns, start and stop sleep patterns, whether sleep is heavy or light, and restlessness. The system 100 may receive inputs from a sleep tracking device for example.

The system 100 may also receive expressions and meditations of gratitude (thought patterns) by, for example, requesting user input of general goals, requesting inputs for user activities performed to help achieve the goals of a user. A numerical weight may be assigned to each activity.

The system 100, in embodiments, may provide a user with practice meditation exercises such as, for example, tracking time spent on meditation, attributing points associated with meditation duration and frequency of meditation. The system 100 may provide a method for recording a quality of the meditation practice.

A physical response may be tracked, such physical responses may include, for example, tracking heartrate, core metrics of a user, respiratory rate, blood pressure and blood oxygen levels.

In embodiments the system 100 may request that a user enter an input that includes why the user is grateful. Such input may be saved and presented to the user in a textual format such as, a gratitude log.

The system 100 is operative to provide a meditation program for a user depending on the condition of the user. The program is tailored to provide meditation guidance depending on goals of the user. For example, one meditation program may be aimed improve a mood of a user. Other programs may include meditation for manifestation and high performance. Manifestation includes, for example, health, happiness, success in personal relationships, successful finances, successful business and self-worth. High performance includes focus, mental clarity, mental calm, physical performance among others.

The system 100 is operative to receive an input from a user regarding their goals associated with the type of meditation. When the user meditates for manifestation or high performance, the system 100 outputs an auditory and vibratory signal such as, for example, music or meditation guidance and vibration associated with improving the manifestation or high-performance factors of the user. In this regard, the auditory signal for manifestation may be similar or dissimilar to the auditory signal for high performance.

A tool for measuring personal energy in terms of frequency of vibration ranging from 1 Hz, which corresponds to the lowest negative vibration, up to 1000 Hz, which corresponds to the maximum positive vibration. Meditation using frequencies from 396 Hz to 963 Hz as auditory sound and vibration in the range of 100 Hz to 250 Hz through the palm of the hand may increase the vibratory frequency of the user.

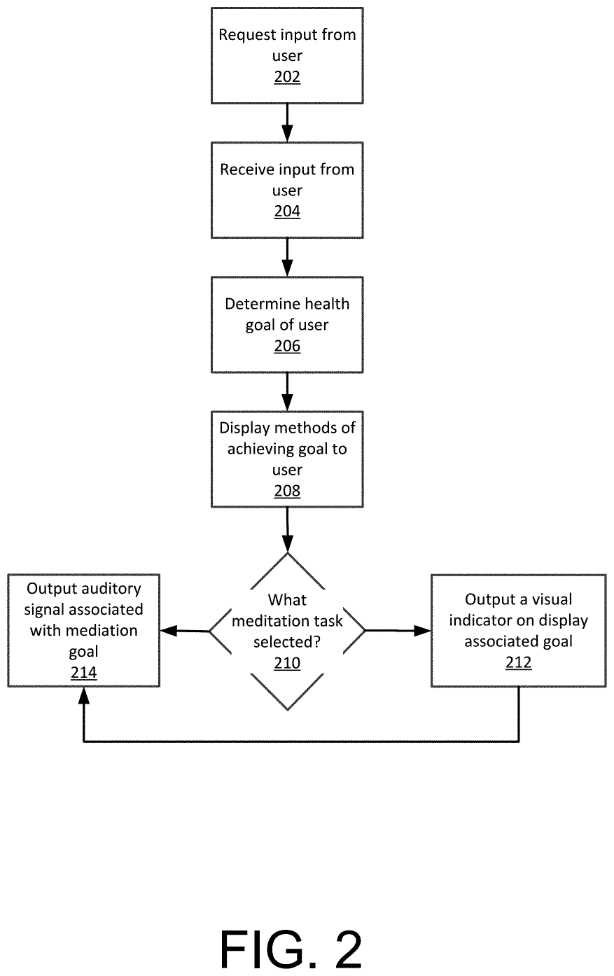

FIG. 2 illustrates a block diagram of a method of operation of the system 100 (of FIG. 1). In this regard, the system 100 requests an input from the user in block 202. The input may include several factors such as, exercise patters, and dietary habits among others as discussed above.

The system 100 receives a corresponding input from the user in block 204. Responsive to receiving an input from the user, the system 100 is operative to determine a wellness goal of the user in block 206. Such goal may include, for example, increased happiness, increased productivity, or unblocking a chakra among others. In block 208, a selection of goals of the user are displayed to the user for example, meditation for improved health, meditation for manifestation, meditation for high performance, or soothing music, which may include a video with audio or another combination of video and audio outputs. In block 206, the system 100 determines a goal for the user based on the input from the user in block 204.

In block 208, the system presents methods of achieving the goal of the user to the user. The goal may include, for example, improving health of a user, reducing stress, or improving the active energy of the user. In block 210 after receiving an input from the user, the system 100 determines which meditation task is selected. In block 212 a visual indicator is displayed that is associated with the selected goal of the user.

The visual indicator may include, for example, a textual recommendation for activities that may be performed by the user to increase the vibrational frequency of the user and improve the therapeutic effects on the user. Such activities may include, for example, meditation or breathing exercises (e.g., box breathing) that may be guided by the system 100. For example, in embodiments the system 100 may present choices and request user input for what type of breathing exercise and the duration of the exercises. Other visual outputs may include, for example, outputting a particular color that corresponds to activities that the user should perform such as, for example, a color of food that corresponds to the output color.

Following block 212 in embodiments, the system 100 may output an auditory signal associated with the meditation goal in block 214. The system 100 may in embodiments output an auditory signal with or without outputting the visual indicator to the user. Such auditory signal may include for example, sounds or music that are optimized to provide a meditative environment conducive to achieving the goals of the user.

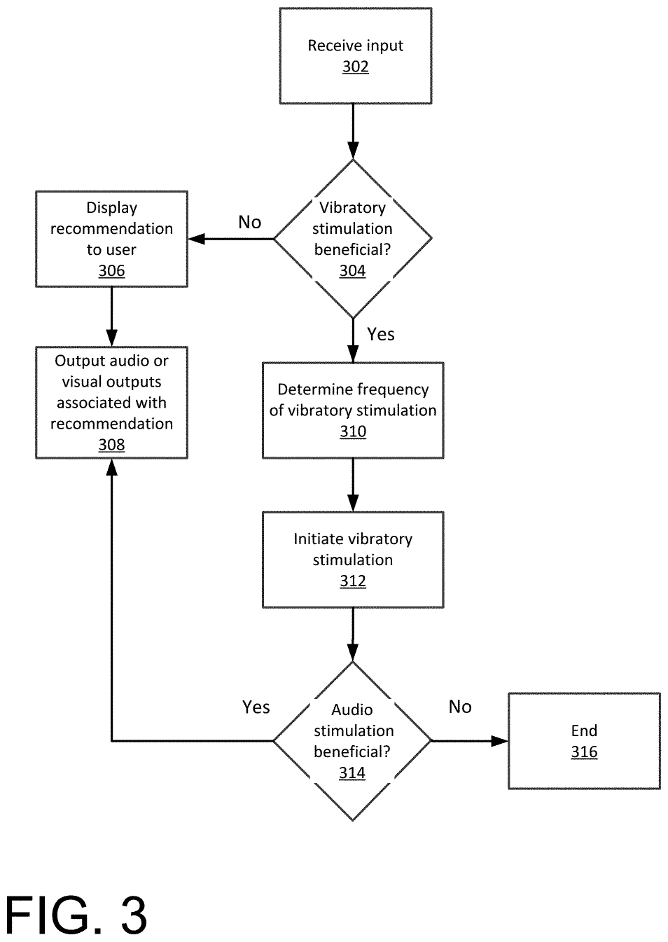

FIG. 3 shows a block diagram of a method that may be performed by the system 100 (of FIG. 1). The method of FIG. 3 may be performed by the system 100 either alone or in combination with the method described above in FIG. 2. In this regard, the method of FIG. 2 may be performed alone.

In operation, the system 100 receives an input 302 from the user that indicates whether the user may benefit from vibrational therapy. In block 304, the system 100 determines whether vibratory stimulation will be beneficial to the user. If no, in block 306, the system 100 displays a recommendation to a user. In block 308, the system outputs an audio signal such as, for example, music (at about 430 Hz), verbal or non-verbal meditation guidance, tones, or other auditory signals. A visual indicator such as a color, patterns or instructions may be displayed to the user in block 308.

If yes in block 304, the system 100 determines a frequency for vibratory stimulation. Such frequency may be associated with the type of therapy determined by the system 100 in block 310. In block 312, the system 100 initiates and outputs a vibratory signal operative to increase the vibrations of the body of the user. The signal may be output by the vibratory portion 120 (in FIG. 1). In embodiments, the vibratory portion 120 may operate at a frequency of about 150 Hz to 250 Hz depending on the type of vibrational stimulation determined in block 310. In block 314 the system 100 determines whether audio stimulation would be beneficial to the user. If yes. an audio or visual output is performed in block 308. In block 316 the process may end.

The vibratory stimulation discussed above may provide a vibration at an increased or decreased frequency. The user may select, or the system 100 may select a frequency that provides a desired therapeutic effect. The vibration may be output as haptic feedback that may be adjustable by the user using, for example, a menu with user selectable vibration frequency settings.

In embodiments the user may, for example, hold the vibratory device in the hand of the user or on a portion of the user such that the vibratory signal of the vibratory portion 120 increases the vibrations of the body in conjugation with auditory sound to improve the meditative therapy for the user.

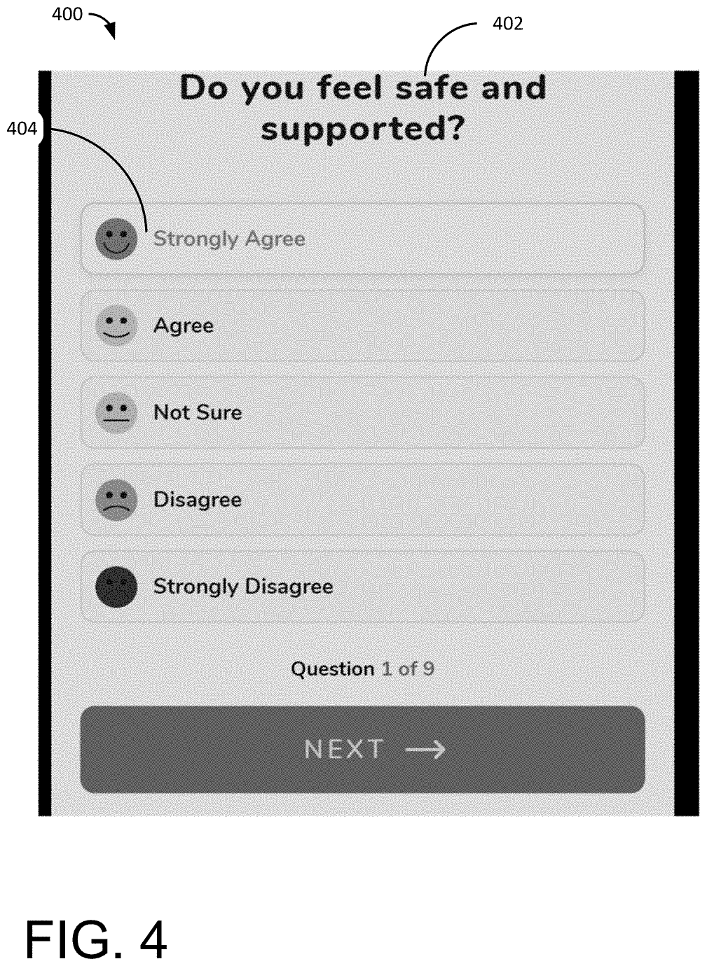

FIGS. 4-19 illustrate a number of exemplary graphical user interfaces that may be presented to a user by the system 100. The user may make selections to various questions and settings in the system 100.

In this regard, FIG. 4 illustrates a graphical representation of a user interface 400 with an example of a question 402 that may be presented to a user, “Do you feel safe and supported?” In embodiments the user may select an answer 404 to the question such as, for example, “Strongly agree,” “Agree,” “Not sure,” “Disagree,” and “Strongly disagree.” Such questions and answers are merely examples and may include any type of question, any number of questions, and any type of answer.

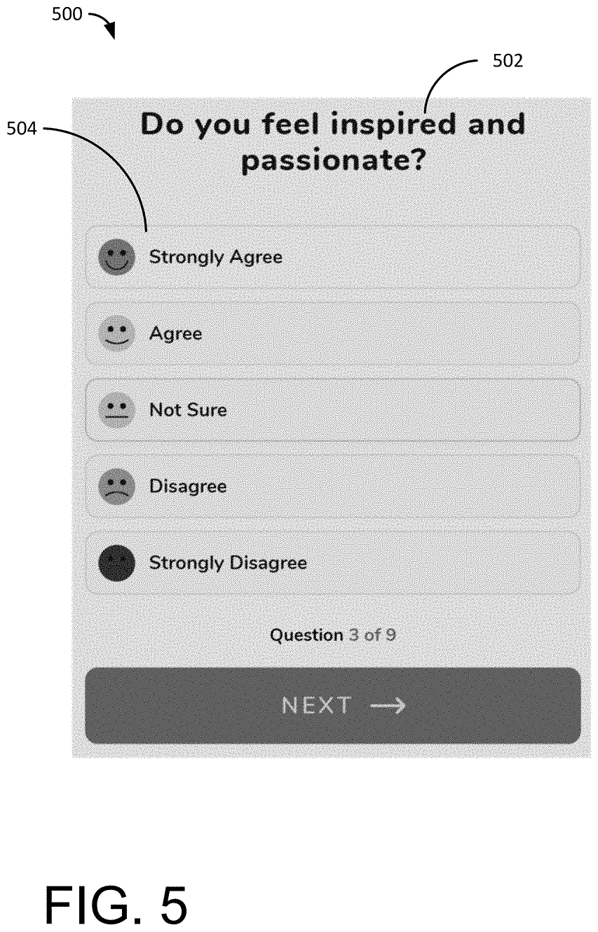

FIG. 5 illustrates another graphical representation 500 of a question 502 that may be presented to a user, “Do you feel inspired and passionate?” Such question provides similar options for answers 504 to the user as discussed above in FIG. 4.

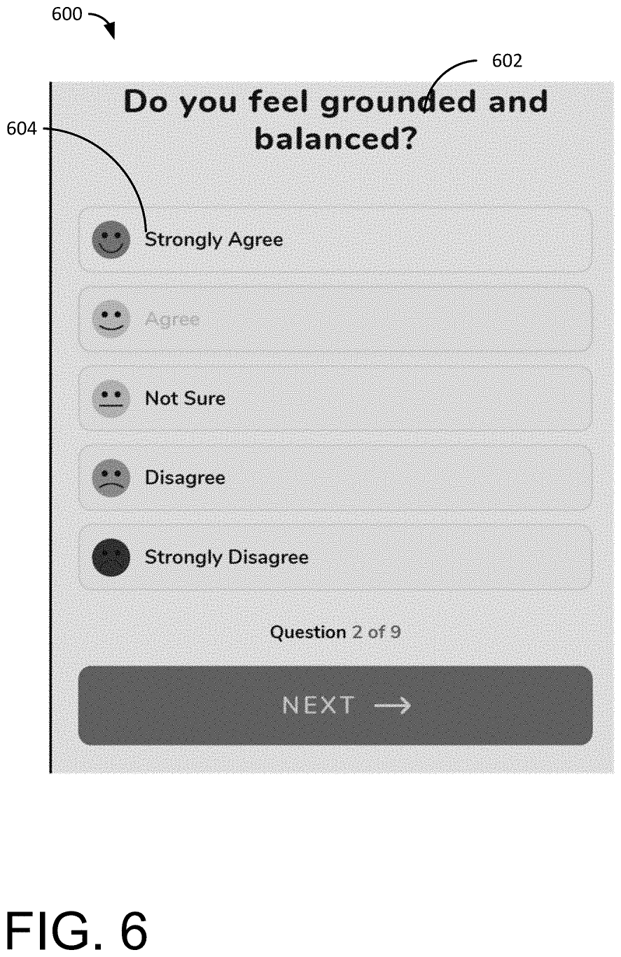

FIG. 6 illustrates yet another graphical representation 600 of a user interface that includes a question 602 that may be presented to a user, “Do you feel grounded and balanced?” Such question provides similar options for answers 604 to the user as discussed above in FIG. 4.

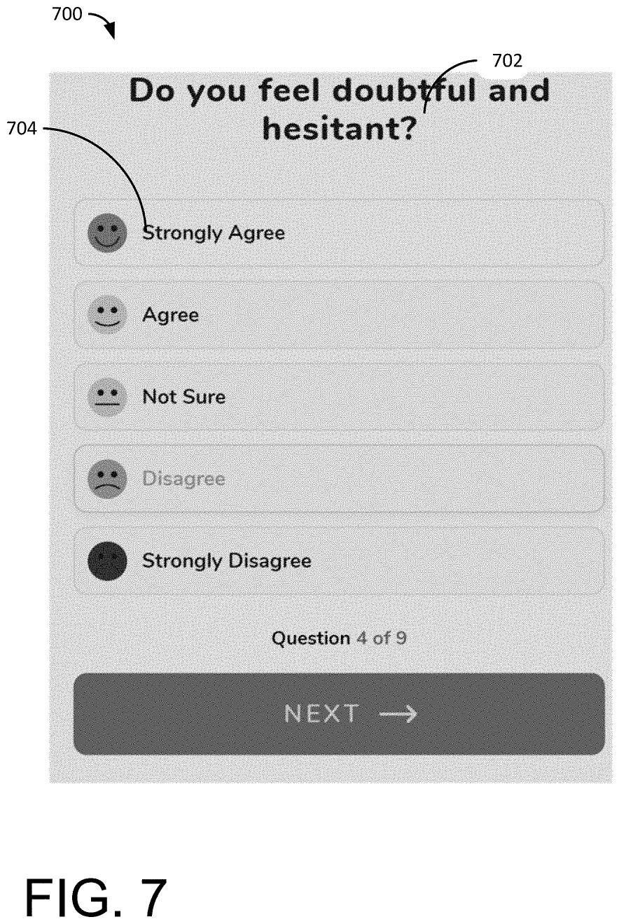

FIG. 7 illustrates yet another graphical representation 700 of a user interface that includes a question 702 that may be presented to a user, “Do you feel doubtful and hesitant?” Such question provides similar options for answers 704 to the user as discussed above in FIG. 4.

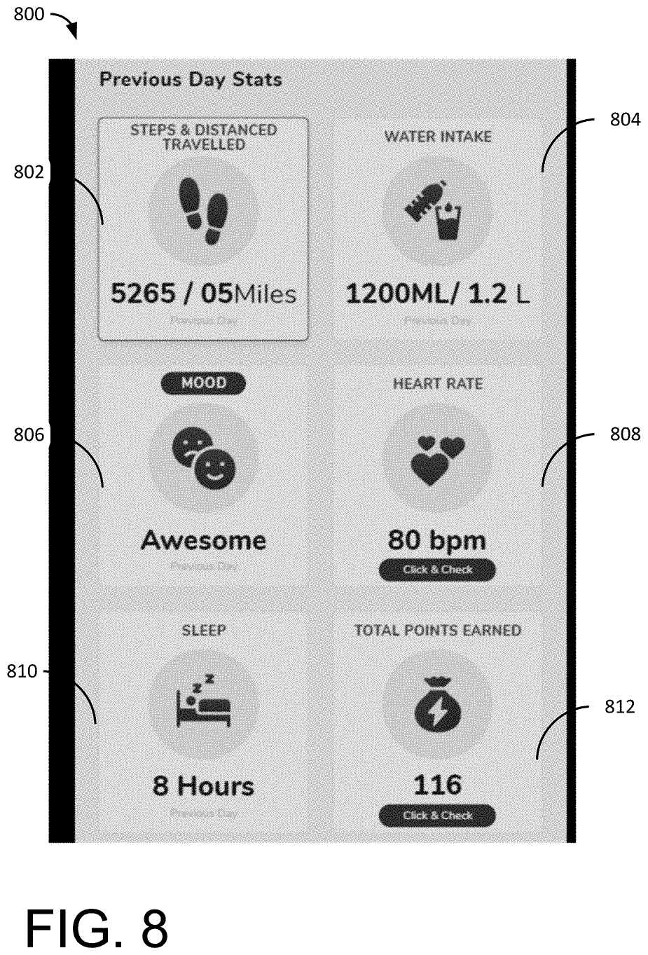

FIG. 8 illustrates a graphical user interface 800 that includes fields that contain statistical information related to several health factors. Such factors include, for example, Steps and Distance traveled by a user 802. Such data may be calculated by a device such as a smartphone or wearable device. Alternatively, a user may manually input a distance traveled for a day. A daily water intake of the user 804. The mood of the user in block 806, the heartrate of the user in 808, and the cumulative sleep of the user in block 810. The scores or data described above may be tabulated to generate a total points earned for a day in block 812.

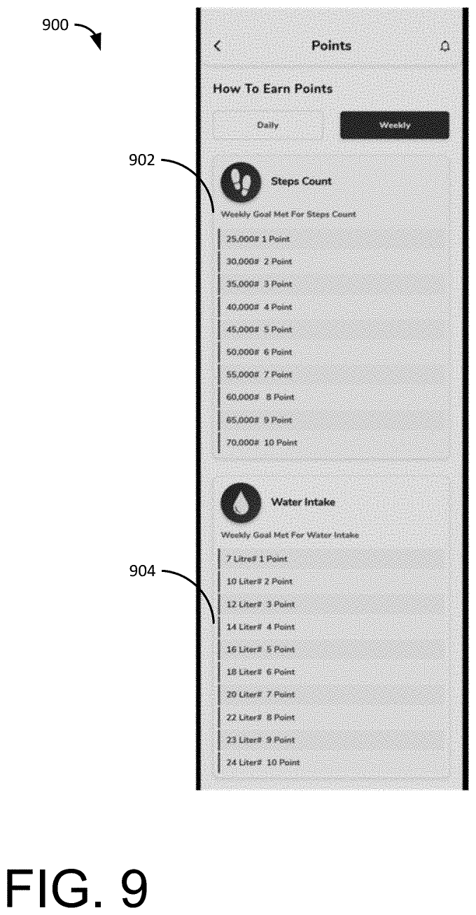

FIG. 9 illustrates a graphical user interface 900 that includes charts that correspond weekly goals for tasks with point values. Thus, for example, in table 902, if a user performs 25,000 steps in a week a point value of 1 point is earned and will be used for an overall score. In block 904, the points corresponding to an amount of weekly water intake are shown. For example, if a user consumes 14 liters of water, the user receives 4 points. Such an arrangement allows users to track their healthy habits and be motivated to improve their weekly score by increasing the amount or frequency of positive tasks such as eating a healthy diet, exercising, and meditating.

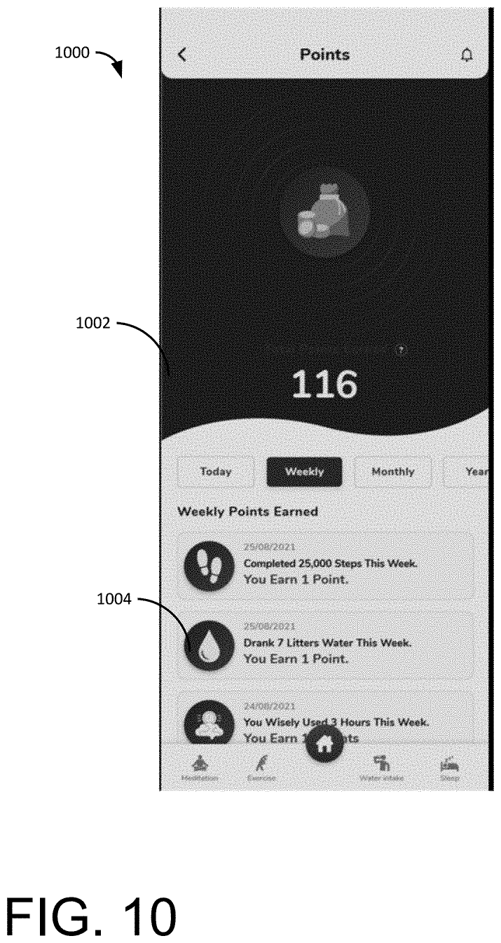

FIG. 10 illustrates an example of a graphical user interface 1000. The user interface 1000 includes a visual indicator of the points earned by a user 1002. The points shown may be selected over a temporal period such as, for example, today, weekly, monthly, or yearly. The points earned for each task over the selected period are shown in detail in block 1004.

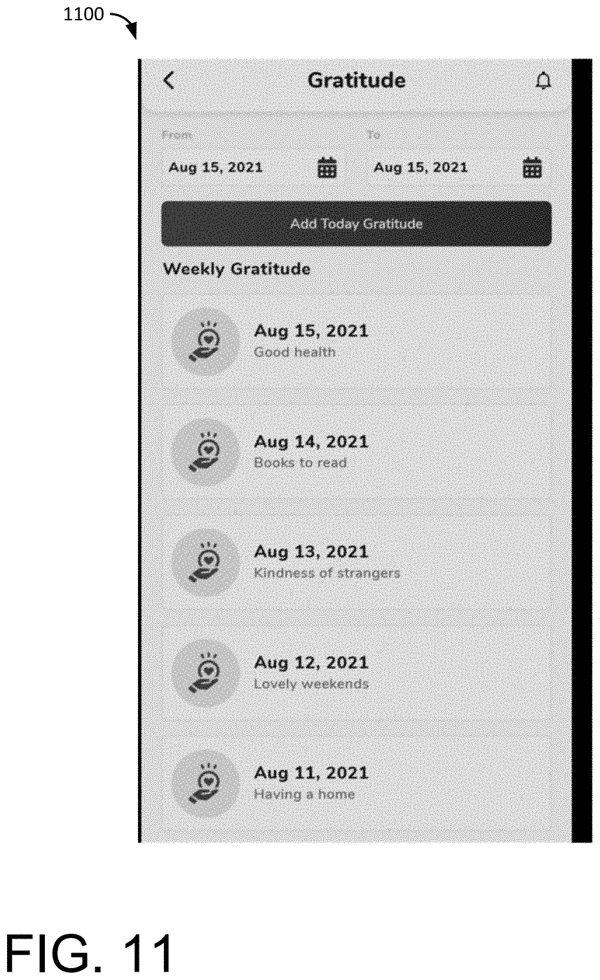

FIG. 11 illustrates an example of a graphical user interface 1100 that may be used to enter something the user is grateful for. For example, a user may enter in a log something that the use is grateful for like “good health,” the kindness of strangers,” or “having a home.” Such entries regarding gratefulness have been known to improve the gratefulness of a user and increase their contentment and happiness.

In operation, the system 100 may use the answers to the questions described in FIGS. 4-6 (described above). To determine a class of meditation that is recommended to the user.

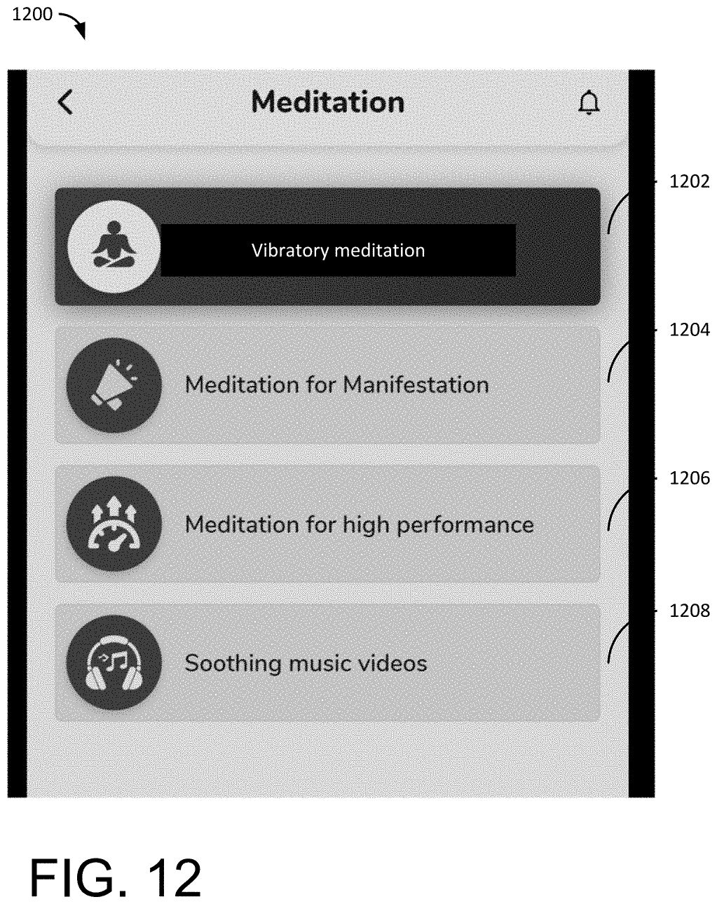

FIG. 12 illustrates a graphical user interface 1200 that includes choices for types of meditation classes that are grouped into categories. A first category of meditation that includes vibratory and auditory therapy at frequencies for the mind and the body in block 1202. A second category of meditation 1204 is meditation for manifestation that includes vibratory and auditory therapy. A third type of meditation 1206 includes meditation for high performance with vibratory and auditory outputs.

Regarding meditation for blocked chakra in block 1202, the therapy has several levels such as levels 1 through 7 that correspond to the questions answered by the user above. An audio response associated with each level in frequency range from about 396 Hz to about 963 Hz has been used to improve the effectiveness of the meditation depending on the needs of the user. A vibrational frequency may be output to improve the therapeutic effects on the user. The vibrational frequency may range from about 100 Hz to about 250 Hz. Soothing music videos 1208 may be another option for a user. Soothing music videos may include music and visual indicators that improve the effectiveness of the therapy on the user.

The table 1 below shows an example of frequency outputs that induce vibratory stimulation and auditory stimulation in a user when the user, for example, holds or rests the vibratory device on the user and listens to music at approximately the auditory frequencies described in the table 1 below. The table below is merely an example, in embodiments other ranges of frequency response may be used by the device 100 (of FIG. 1).

TABLE 1

| Level | Vibratory frequency | Auditory frequency |
|---|---|---|
| 1 | 100 Hz | 350 Hz-399 Hz |
| 2 | 100 Hz | 400 Hz-417 Hz |
| 3 | 120 HZ | 450 Hz-528 Hz |
| 4 | 150 HZ | 600 Hz-693 Hz |
| 5 | 180 HZ | 700 Hz-741 Hz |
| 6 | 200 HZ | 750 Hz-852 Hz |
| 7 | 250 HZ | 900 Hz-963 Hz |

Meditation for manifestation is option 1204. Meditation for manifestation includes improving the health, happiness, success in personal relationships in financial and business, and self-worth. Meditation for manifestation provides music or sounds that include music and a guided meditation track with vibration therapy. The guided meditation track may include a voice or other auditory indicators to improve the wellbeing of the user.

Meditation for high performance is operative to improve focus, mental clarity, mental calm, and physical performance. For meditation for high performance, the user is presented with music and/or guided meditation that is associated with a meditative high-performance goal with vibrational therapy.

Another option presented to the user is, “soothing music videos.” Such soothing music and videos may be used by a user to meditate or as ambient sound and visual stimulation.

Based on the answers from the user in 800, a color or other visual indicator may be presented to the user to remind the user to perform a task such as, for example, eating a particular diet or performing a particular task to improve the health of the user.

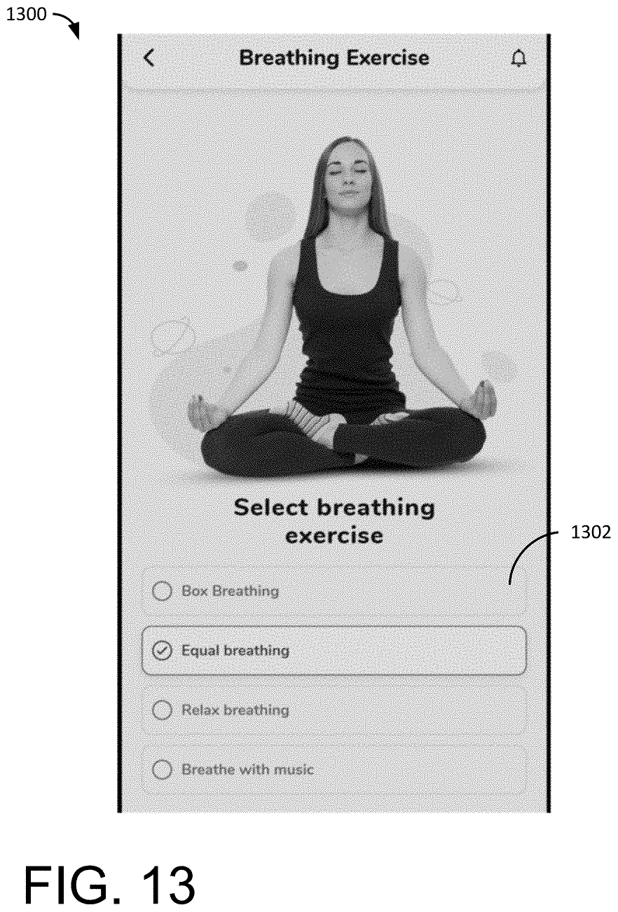

FIG. 13 illustrates a graphical user interface 1300 that includes a selection of breathing exercises. The breathing exercises may be used as part of the recommendation based on the answers to the questions above. Breathing exercises 1302 may include, for example, box breathing, equal breathing, relaxation breathing, or breathing with an auditory output at an interval to calm the body and the mind.

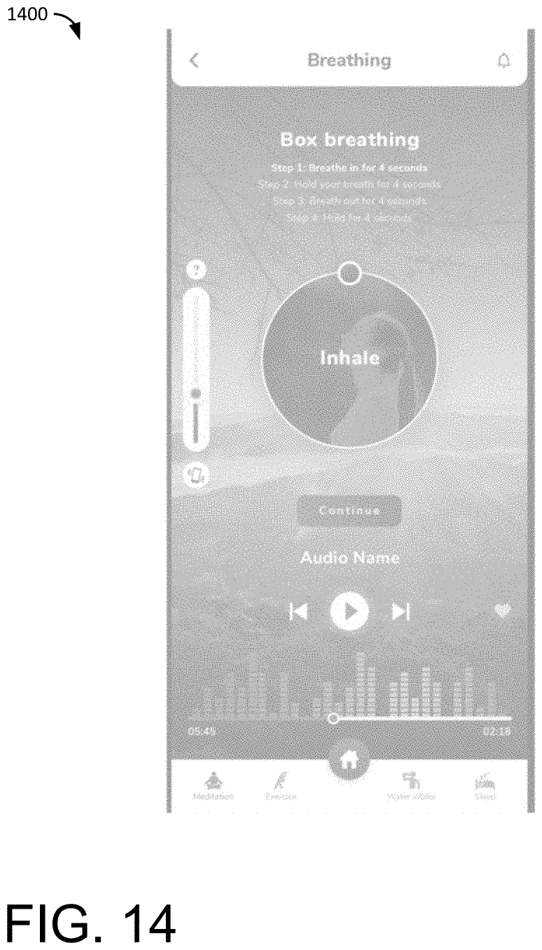

FIG. 14 illustrates a graphical user interface 1400 of a box breathing task. The box breathing interface provides breathing guidance and controls of an audio track that may facilitate box breathing such as, for example holding one's breath for a portion of time, releasing the breath for a portion of time and inhaling for a portion of time.

Embodiments described herein include a device, system, and method operative to determine and identify a number of tasks directed to improving the health of a user and prescribe a regimen that improves the effectiveness of such regimen. In embodiments, the system 100 is operative to guide a user in improving their wellness.

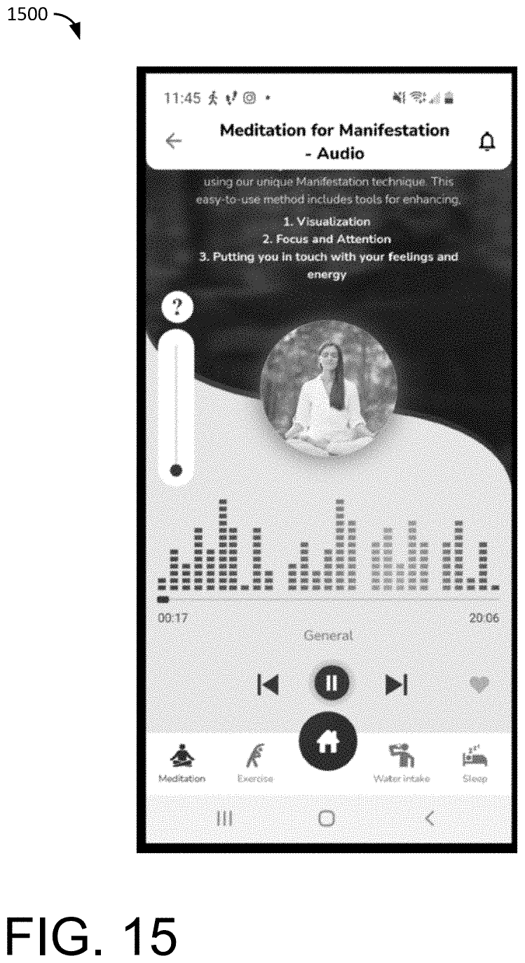

FIG. 15 illustrates a graphical user interface 1500 of the system 100 (of FIG. 1). The interface includes an output during the meditation for manifestation task. The interface includes meditation instructions, an audio track that may include music, sounds or verbal meditation instructions and vibrational therapy tailored to meet the manifestation meditation goal.

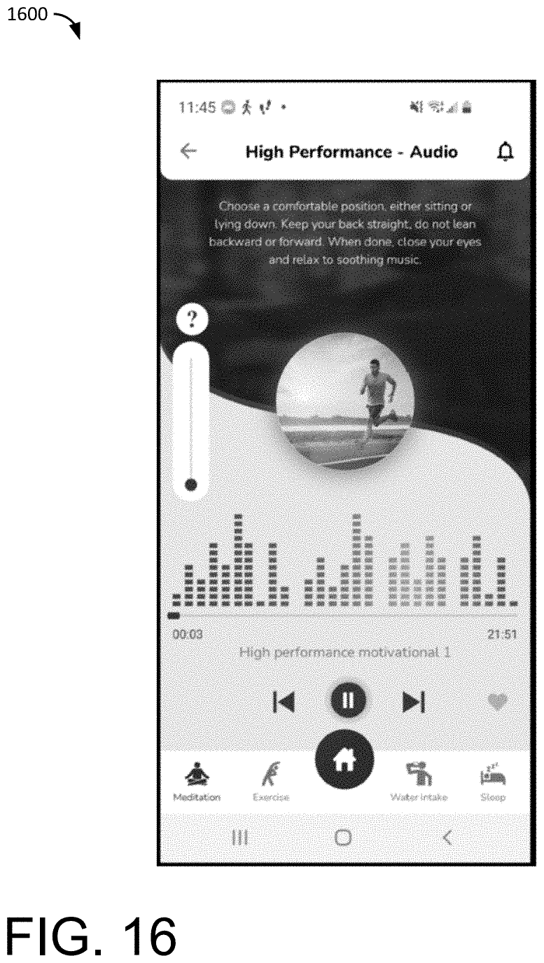

FIG. 16 illustrates a graphical user interface 1600 of the system 100 (of FIG. 1). The interface includes an output during the meditation for high performance task. The interface includes meditation instructions, an audio track that may include music, sounds or verbal meditation instructions and vibrational therapy tailored to reach the high-performance meditation goal.

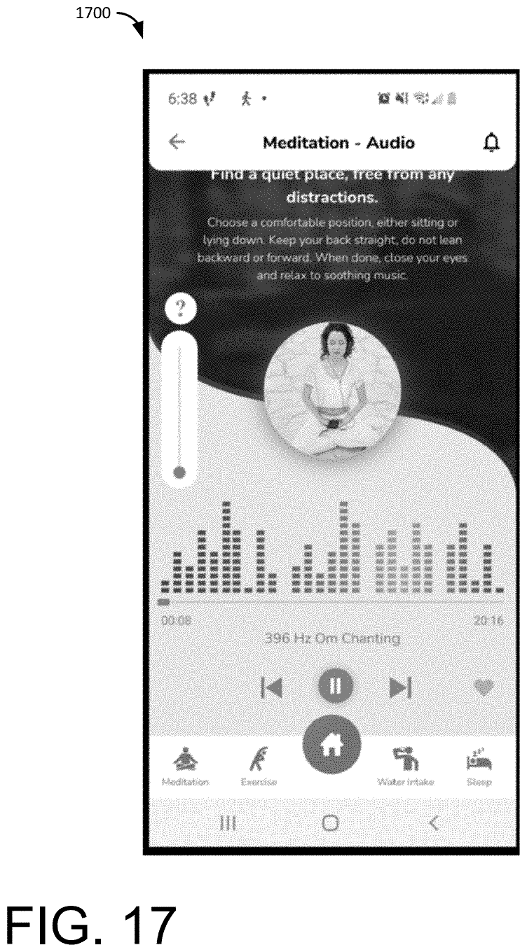

FIG. 17 illustrates a graphical user interface 1700 of the system 100 (of FIG. 1). The interface includes an audio output option during the meditation task. The interface includes meditation instructions, an audio track that may include music, sounds, or verbal meditation instructions and vibrational therapy. In embodiments vibrational therapy may be tailored to reach the meditation goal.

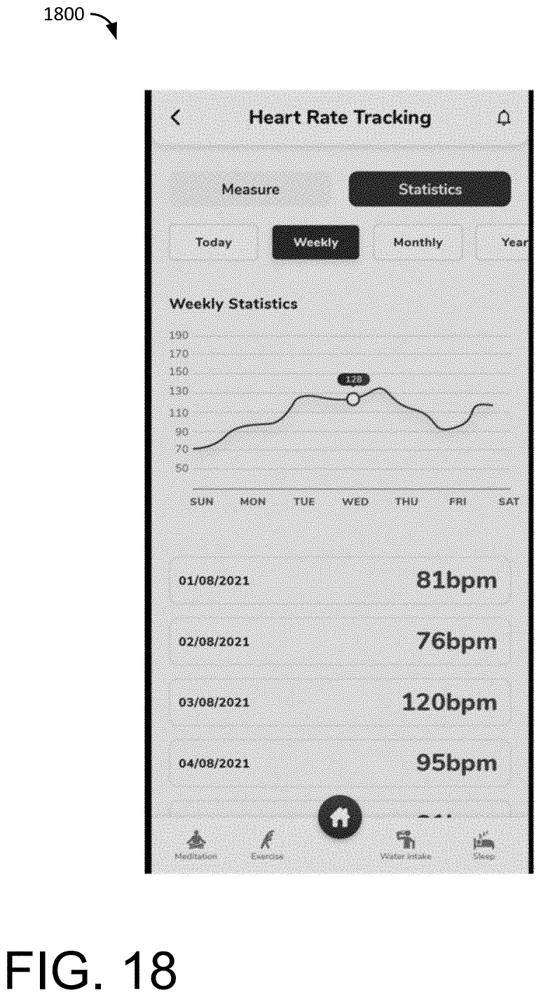

FIG. 18 illustrates a heart rate tracking interface 1800. The heart rate tracking may be performed by, for example a wearable device or another suitable heart rate tracking device or method. The heart rate tracking feature provides feedback regarding a higher or lower heart rate during a period of time. The heart rate tracking can inform the system 100 and the user that the tasks assigned for increasing their health in particularly lowering their heartrate has been successful. The therapies described above often have positive effects on the heart rate of a user.

Similar to tracking heartrate, the use of other health metrics such as, for example, blood pressure, blood oxygen level, strength, endurance, alertness, cognitive ability may all be used to determine the effectiveness of a particular set of tasks performed by the user.

In this regard, tracking the metrics associated with good health may be used to determine whether the particular tasks performed by a user have been improved by performing the tasks recommended by the system 100.

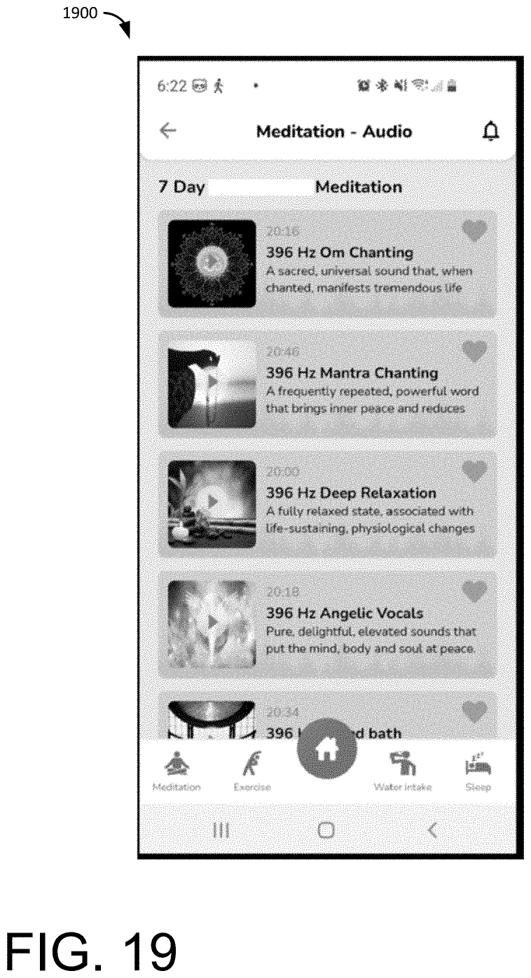

FIG. 19 illustrates a graphical user interface 1900 that includes daily meditation tasks for the user to achieve a meditation goal of the user. The interface 1900 includes, for example, a list of daily meditation exercises that may include auditory outputs for the user and vibrational therapy in embodiments.

The present invention can be a system, a method, and/or a computer program product at any possible technical detail level of integration. The computer program product can include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for improving health of a user, the method comprising:
receiving an answer to a question from the user;
identifying a first category of therapy associated with the answer to the question;
displaying a visual indicator associated with the first category to the user; and
outputting guidance for the user including tasks operative to improve the health of the user, the guidance including a vibratory output associated with the first category operative to improve the health of the user.

2. The method of claim 1, wherein the guidance includes a diet of the user.

3. The method of claim 1, wherein the guidance includes a visual indicator with recommendations improve the health of the user.

4. The method of claim 1, wherein the guidance includes a breathing exercise.

5. The method of claim 1, wherein the visual indicator includes a color associated to improve the health of a user.

6. The method of claim 1, wherein the vibratory output is at a frequency of about 100 Hz to about 250 Hz.

7. The method of claim 1, wherein a vibratory device vibrates at a frequency of about 150 Hz to about 250 Hz.

8. The method of claim 1, wherein a vibratory device includes a smartphone.

9. The method of claim 1, further comprising identifying a second category of therapy responsive to receiving a second answer to a question from the user, the second category operative to output an audible signal operative to improve a meditative state of the user and increase the health of the user.

10. The method of claim 9, wherein the audible signal includes music and verbal signals.

11. A system for improving health of a user, the system comprising:
a memory;
a display; and
a processor operative to:
receive an answer to a question from the user;
identify a first category of therapy associated with the answer to the question;
display a visual indicator associated with the first category to the user; and
output a guidance for the user including tasks operative to improve the health of the user, the guidance including a vibratory output associated with the first category operative to improve the health of the user.

12. The system of claim 11, wherein the guidance includes a diet of the user.

13. The system of claim 11, wherein the guidance includes a visual indicator with recommendations improve the health of the user.

14. The system of claim 11, wherein the guidance includes a breathing exercise.

15. The system of claim 11, wherein the visual indicator includes a color associated to improve the health of a user.

16. The system of claim 11, wherein the vibratory output is at a frequency of about 100 Hz to about 250 Hz.

17. The system of claim 11, further comprising a vibratory device that vibrates at a frequency of about 150 Hz to about 250 Hz.

18. The system of claim 11, wherein the vibratory device includes a smartphone.

19. The system of claim 11, further comprising identifying a second category of therapy responsive to receiving a second answer to a question from the user, the second category operative to output an audible signal operative to improve a meditative state of the user and increase the health of the user.

20. A method for improving health of a user, the method comprising:
receiving an answer to a question from the user;
identifying a first category of therapy associated with the answer to the question;
displaying a visual indicator associated with the first category to the user, the visual indicator including a color recommendation of a diet for the user, and a meditative therapy recommendation that includes vibrational therapy at about 100 Hz to 250 Hz; and
outputting guidance for the user including tasks operative to improve the health of the user, the guidance including a vibratory and auditory output associated with the first category operative to improve the health of the user.
